(19) United States
(12) Patent Application Publication
(10) Pub. No.: US 2014/0223987 A1
(43) Pub. Date: Aug. 14, 2014

(54) APPARATUS FOR DETECTING CYLINDER AIR-FUEL RATIO IMBALANCE ABNORMALITY OF MULTI-CYLINDER INTERNAL COMBUSTION ENGINE

(71) Applicants: Tatsuro Shimada, Toyota-shi (JP); Isao Nakajima, Nisshin-shi (JP); Toshihiro Kato, Toyota-shi (JP); Leuth Insixiengmai, Nagoya-shi (JP); Yoshihisa Oda, Toyota-shi (JP); Masashi Hakariya, Nagoya-shi (JP)

(72) Inventors: Tatsuro Shimada, Toyota-shi (JP); Isao Nakajima, Nisshin-shi (JP); Toshihiro Kato, Toyota-shi (JP); Leuth Insixiengmai, Nagoya-shi (JP); Yoshihisa Oda, Toyota-shi (JP); Masashi Hakariya, Nagoya-shi (JP)

(73) Assignee: TOYOTA JIDOSHA KABUSHIKI KAISHA, Toyota-shi (JP)

(21) Appl. No.: 14/166,024

(22) Filed: Jan. 28, 2014

(30) Foreign Application Priority Data
Feb. 13, 2013 (JP) 2013-025824

(51) Int. Cl. G01N 33/00

(52) U.S. Cl. CPC G01N 33/0006
USPC 73/1.06

(57) ABSTRACT

Provided is an apparatus for detecting cylinder air-fuel ratio imbalance abnormality. The apparatus is provided with an abnormality detecting unit that detects a cylinder air-fuel ratio imbalance abnormality by comparing a value of a parameter correlated with a degree of fluctuation in the air-fuel ratio sensor output to an abnormality threshold value, and a correcting unit that corrects at least one of the value of the parameter or the abnormality threshold value on the basis of atmospheric pressure. An amount of correction performed by the correcting unit is modified according to engine load.

### CROSS-REFERENCES TO RELATED APPLICATIONS

This application claims the benefit of Japanese Patent Application No. 2013-025824, filed Feb. 13, 2013 which is hereby incorporated by reference herein in its entirety.

### BACKGROUND OF THE INVENTION

1. Field of the Invention

The present invention relates to an apparatus for detecting an imbalance abnormality in the cylinder air-fuel ratio of a multi-cylinder internal combustion engine, and more particularly, to an apparatus that detects a major imbalance of the air-fuel ratio among cylinders in a multi-cylinder internal combustion engine.

2. Description of the Related Art

In an internal combustion engine equipped with an exhaust purification system that utilizes a catalyst, pollutants in exhaust are efficiently purified by the catalyst, and thus control of the mixing proportion between air and fuel in the air-fuel mixture that is burned in the internal combustion engine, or control of the air-fuel ratio, is essential. In order to perform such control of the air-fuel ratio, an air-fuel ratio sensor is provided in the exhaust passage of the internal combustion engine, and a feedback control is carried out to match a detected air-fuel ratio to a target air-fuel ratio.

Meanwhile, in a multi-cylinder internal combustion engine, since ordinarily the same control amount for all cylinders is used to conduct the air-fuel ratio control, the actual air-fuel ratio may become imbalanced across cylinders, even if the air-fuel ratio control is executed. If the degree of the imbalance at this point is small, it would be absorbable by the air-fuel ratio feedback control, and pollutants in the exhaust may still be purified by the catalyst. Thus, the imbalance does not affect exhaust emissions, and does not pose a particular problem.

However, if the air-fuel ratio among cylinders is greatly imbalanced due to factors such as a failure of the fuel injection system in some of the cylinders for example, the imbalance causes worsened exhaust emissions, and poses a problem. It is desirable to detect such large air-fuel ratio imbalances that worsen exhaust emissions as an abnormality. Particularly in the case of an internal combustion engine for an automobile, in order to prevent vehicle travel with worsened exhaust emissions from occurring, there is demand for onboard detection of cylinder air-fuel ratio imbalance abnormality, and recently there has also been movement to legally enforce such a feature.

In order to detect cylinder air-fuel ratio imbalance abnormality, the device described in Japanese Patent Laid-Open No. 2012-092803, for example, uses the output of an air-fuel ratio sensor placed in a junction part of an exhaust pipe. The device is configured to compare the value of a parameter correlated with the degree of fluctuation in the output of the air-fuel ratio sensor against a predetermined abnormality threshold value, and determine that an imbalance abnormality has occurred in the case of exceeding the abnormality threshold value.

Meanwhile, in a multi-cylinder internal combustion engine, if the atmospheric pressure changes, the degree of exhaust interference also changes, and the output of an air-fuel ratio sensor becomes different. Consequently, determining abnormality by comparing against a fixed abnormality threshold value makes precision of determination inconsistent. For this reason, in order to eliminate the influences of atmospheric pressure and improve the detection precision, the device in the Japanese Patent Laid-Open No. 2012-092803 is configured to correct, on the basis of the atmospheric pressure, at least one of either the value of the parameter correlated with the degree of fluctuation in the output of the air-fuel ratio sensor, or the abnormality threshold value.

However, the degree of exhaust interference among cylinders also changes depending on the load. For this reason, determining abnormality without taking exhaust interference into account makes precision of determination inconsistent.

Accordingly, the present invention was devised in light of the above circumstances, and an object thereof is to provide an apparatus for detecting a cylinder air-fuel ratio imbalance abnormality of a multi-cylinder internal combustion engine that may further improve precision of detection and prevent misdetections.

### SUMMARY OF THE INVENTION

One mode of the present invention is an apparatus for detecting cylinder air-fuel ratio imbalance abnormality of a multi-cylinder internal combustion engine, provided with: one or multiple air-fuel ratio sensors installed in an exhaust passage of the multi-cylinder internal combustion engine;

an abnormality detecting unit that detects a cylinder air-fuel ratio imbalance abnormality by comparing a value of a parameter correlated with a degree of fluctuation in the air-fuel ratio sensor output to an abnormality threshold value; and

a correcting unit that corrects at least one of the value of the parameter or the abnormality threshold value on the basis of atmospheric pressure;

wherein an amount of correction performed by the correcting unit is modified according to a load of the multi-cylinder internal combustion engine.

In a preferred mode, the correcting unit corrects the value of the parameter, while the amount of correction is modified in a direction such that an absolute value of the degree of fluctuation increases as the load increases.

In another preferred mode, the correcting unit corrects the value of the abnormality threshold value, while the amount of correction is modified in a direction such that an absolute value of the degree of fluctuation decreases as the load increases.

In another preferred mode, multiple, mutually differing correcting units are provided, and the mutually differing correcting units are applied to one part and another part of a plurality of cylinders.

Further features of the present invention will become apparent from the following description of exemplary embodiments (with reference to the attached drawings).

### BRIEF DESCRIPTION OF THE DRAWINGS

FIG. 1 is a schematic diagram of an internal combustion engine according to a first embodiment of the present invention;

FIG. 2 is a graph illustrating output characteristics of a pre-catalyst sensor and a post-catalyst sensor;

FIG. 3 is a timing chart illustrating change in an air-fuel ratio detected by a pre-catalyst sensor in the case where, under normal pressure, only cylinders #1, #3, #5, and #7 are rich, respectively, while the other three cylinders are stoichiometric;

FIG. 4 is a timing chart corresponding to FIG. 3 in a low-pressure environment;

FIG. 5 is a timing chart that schematically illustrates a relationship between fluctuation in the output of a pre-catalyst sensor within one engine cycle, and a fluctuation parameter;

FIG. 6 is a table illustrating exemplary settings for a correction coefficient map;

FIG. 7 is a graph corresponding to the correction coefficient map in FIG. 6;

FIG. 8 is a flowchart illustrating a routine for detecting a cylinder air-fuel ratio imbalance abnormality in the first embodiment; and

FIG. 9 is a flowchart illustrating a routine for detecting a cylinder air-fuel ratio imbalance abnormality in a second embodiment.

### DESCRIPTION OF THE EMBODIMENTS

Hereinafter, exemplary embodiments of the present invention will be described on the basis of the attached drawings.

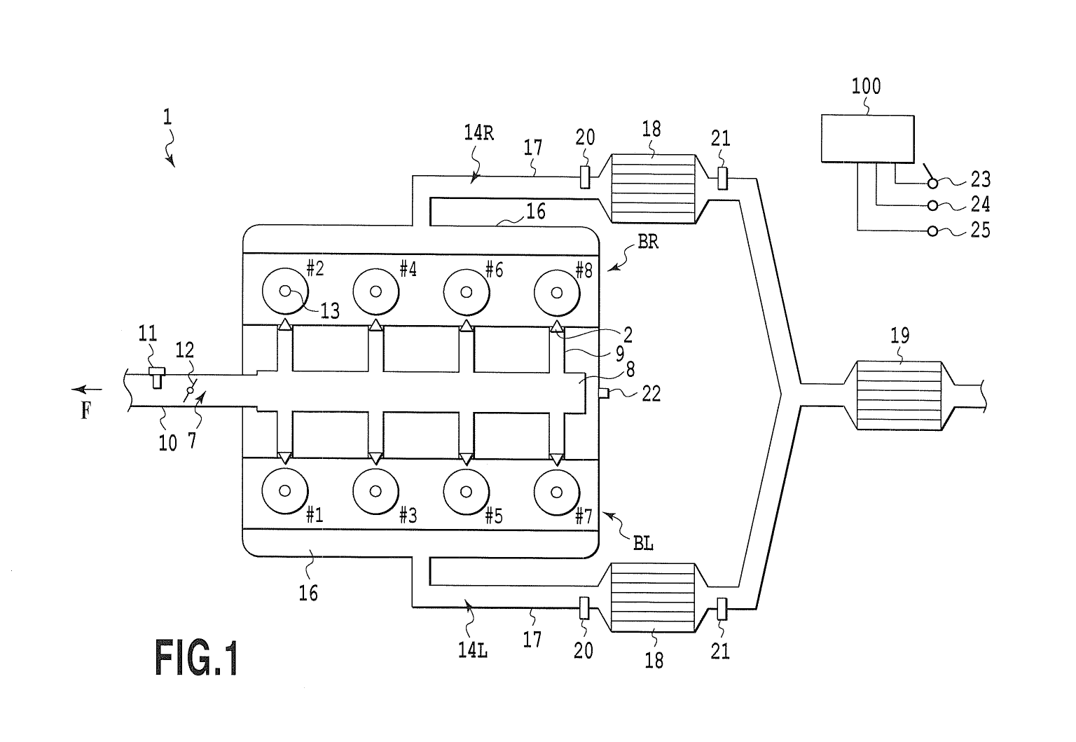

FIG. 1 schematically illustrates an internal combustion engine according to a first embodiment of the present invention. The internal combustion engine 1 as illustrated is a V-type, 8-cylinder, spark-ignited internal combustion engine (gasoline engine). When viewing the engine in a forward direction F, the engine 1 includes a right bank BR on the right side, and a left bank BL on the left side. On the left bank BL are provided the odd-numbered cylinder, or in other words the cylinders #1, #3, #5, and #7 in that order from the front, while on the right bank BR are provided the even-numbered cylinders, or in other words the cylinders #2, #4, #6, and #8 in that order from the front. The odd-numbered cylinders #1, #3, #5, and #7 form a first cylinder group, while the even-numbered cylinders #2, #4, #6, and #8 form a second cylinder group.

In addition, an injector (fuel injection valve) 2 is provided for each cylinder. In other words, the injectors 2 inject fuel into the intake passage of a corresponding cylinder, and particularly into an intake port (not illustrated). Also, each cylinder is provided with a spark plug 13 for igniting the air-fuel mixture inside the cylinder.

The intake passage 7 for introducing intake air into each cylinder includes, besides the above intake ports, a surge tank 8 that acts as a junction part, an intake manifold 9 that joins the intake port of each cylinder to the surge tank 8, and an intake pipe 10 on the upstream side of the surge tank 8. The intake pipe 10 is provided with an airflow meter 11 and an electronically controlled throttle valve 12 in an order from the upstream side. The airflow meter 11 outputs a signal whose magnitude corresponds to the intake flow rate.

A right exhaust passage 14R is provided for the right bank BR, while a left exhaust passage 14L is provided for the left bank BL. The right and left exhaust passages 14R and 14L converge on the upstream side of a downstream catalyst 19. Since the configuration of the exhaust systems upstream to this convergence position is the same for both banks, herein only the right bank BR side will be explained, whereas the left bank BL side is labeled with the same signs in the drawing, and omitted from explanation.

The right exhaust passage 14R includes an exhaust port (not illustrated) for each of the cylinders #2, #4, #6, and #8, an exhaust manifold 16 that collects exhaust gas from these exhaust ports, and an exhaust pipe 17 installed on the downstream side of the exhaust manifold 16. In addition, an upstream catalyst 18 is provided in the exhaust pipe 17. On the upstream and downstream sides of the catalyst 18 (immediately before and immediately after), there are respectively installed a pre-catalyst sensor 20 and a post-catalyst sensor 21, which are air-fuel ratio sensors for detecting the air-fuel ratio of exhaust gas. In this way, one each of a shared upstream catalyst 18, pre-catalyst sensor 20, and post-catalyst sensor 21 are respectively provided for multiple cylinders (or a cylinder group) belonging to one of the banks. Note that it is also possible to not converge the right and left exhaust passages 14R and 14L, and provide individual downstream catalysts 19. Two or more air-fuel ratio sensors may also be provided for each of the exhaust passages 14R and 14L. Alternatively, one or multiple air-fuel ratio sensors may be provided on the downstream side of the convergence point of both exhaust passages 14R and 14L.

The engine 1 is additionally provided with an electronic control unit (hereinafter referred to as ECU) 100 that acts as a control unit and a detection unit. The ECU 100 is a commonly known microprocessor, and includes components such as a CPU, ROM, RAM, input/output ports, and a storage device, while none of which are illustrated. Besides the above airflow meter 11, pre-catalyst sensor 20, and post-catalyst sensor 21, various other sensors, such as a crank position sensor 22 for detecting the crank angle or a position in the rotary direction of the engine 1, an accelerator position sensor 23 for detecting the accelerator position, a water temperature sensor 24 for detecting the temperature of engine cooling water, and an atmospheric pressure sensor 25 which is positioned inside the case housing the ECU 100 and which detects the atmospheric pressure, are electrically connected to the ECU 100 via an A/D converter or the like (not illustrated). The ECU 100, on the basis of operating input from the driver and detected values from various sensors, controls the injectors 2, the spark plugs 13, and the throttle valve 12 to obtain a desired output, thereby controlling the fuel injection rate, the fuel injection timing, the ignition timing, the throttle position, and the like.

The throttle valve 12 is provided with a throttle position sensor (not illustrated), and a signal from the throttle position sensor is sent to the ECU 100. The ECU 100 ordinarily performs a feedback control to set the position of the throttle valve 12 (the throttle position) to a position determined according to the accelerator position.

Also, the ECU 100, on the basis of a signal from the airflow meter 11, detects the amount of intake airflow per unit time, or in other words, the intake airflow rate. The ECU 100 then detects the load on the engine 1 on the basis of at least one of the detected accelerator position, the throttle position, and the intake airflow rate.

The ECU 100, on the basis of a crank pulse signal from the crank position sensor 22, detects the crank itself while also detecting the rotation rate of the engine 1. Herein, “rotation rate” refers to the number of revolutions per unit time, and is synonymous with the rotational speed. In the present embodiment, “rotation rate” refers to the number of revolutions per minute, i.e. “rpm”.

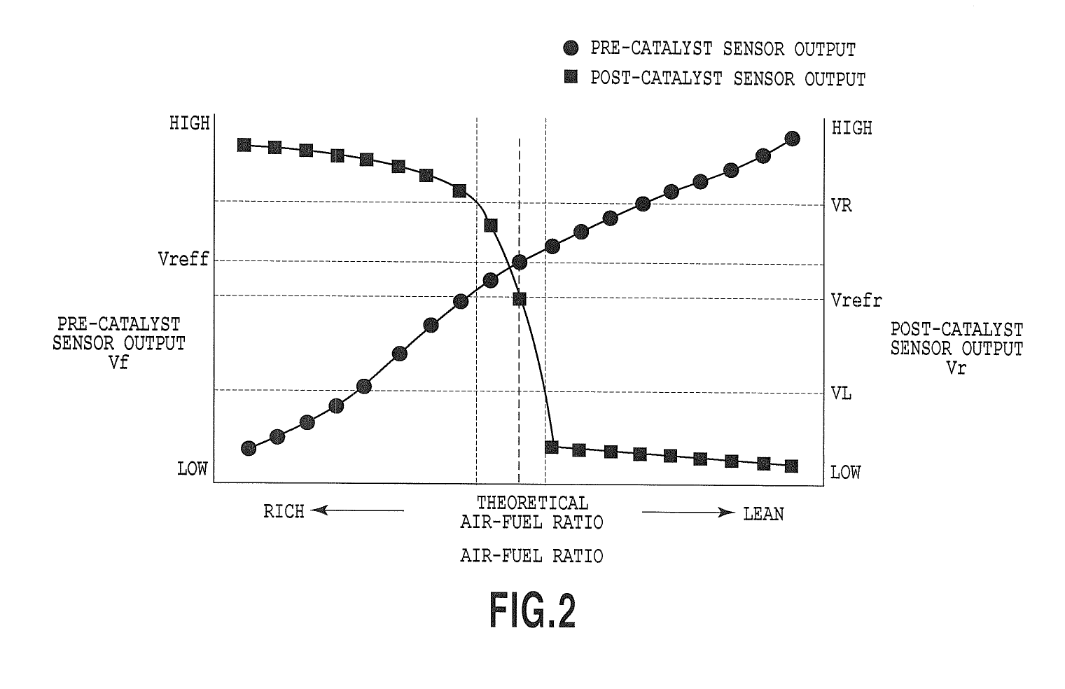

The pre-catalyst sensor 20 is made up of what is called a wide-range air-fuel ratio sensor, and is capable of continuously detecting the air-fuel ratio over a comparatively wide range. FIG. 2 illustrates output characteristics of the pre-catalyst sensor 20. As illustrated, the pre-catalyst sensor 20 outputs an electrical signal Vf whose magnitude is proportional to the detected exhaust air-fuel ratio (the pre-catalyst air-fuel ratio A/Ff). When the exhaust air-fuel ratio is stoichiometric (the theoretical air-fuel ratio, for example A/F=14.5), the output voltage is Vreff (approximately 3.3 V, for example).

On the other hand, the post-catalyst sensor 21 is what is called an O₂ sensor, and has the characteristic of its output value varying sharply about the stoichiometric value. FIG. 2 illustrates output characteristics of the post-catalyst sensor 21. As illustrated, the output voltage when the exhaust air-fuel ratio (the post-catalyst air-fuel ratio A/Fr) is stoichiometric, or in other words the stoichiometric-equivalent value, is Vrefr (0.45 V, for example). The output voltage of the post-catalyst sensor 21 varies within a predetermined range (from 0 V to 1 V, for example). Generally, when the exhaust air-fuel ratio is leaner than stoichiometric, the output voltage Vr of the post-catalyst sensor falls below the stoichiometric-equivalent value Vrefr, and when the exhaust air-fuel ratio is richer than stoichiometric, the output voltage Vr of the post-catalyst sensor rises above the stoichiometric-equivalent value Vrefr.

The upstream catalyst 18 and the downstream catalyst 19 are both made up of a three-way catalyst, and when the air-fuel ratio A/F of respectively inflowing exhaust gas is near-stoichiometric, simultaneously purify the pollutants NOx, HC, and CO in the exhaust. The air-fuel ratio window in which these three pollutants may be efficiently purified is comparatively narrow.

Accordingly, during ordinary engine operation, the ECU 100 executes an air-fuel ratio feedback control (stoichiometric control) in order to keep the air-fuel ratio of exhaust gas flowing into the upstream catalyst 18 near-stoichiometric. This air-fuel ratio feedback control is made up of a primary air-fuel ratio control (primary air-fuel ratio feedback control) that performs feedback control of the air-fuel ratio of the air-fuel mixture (specifically, the fuel injection rate) so that the exhaust air-fuel ratio detected by the pre-catalyst sensor 20 becomes stoichiometric at a predetermined target air-fuel ratio, and an auxiliary air-fuel ratio control (auxiliary air-fuel ratio feedback control) that performs feedback control of the air-fuel ratio of the air-fuel mixture (specifically, the fuel injection rate) so that the exhaust air-fuel ratio detected by the post-catalyst sensor 21 becomes stoichiometric.

In this way, in the present embodiment, the reference value of the air-fuel ratio is stoichiometric, and the fuel injection rate corresponding to stoichiometric (referred to as a stoichiometric-equivalent value) is the reference value for the fuel injection rate. However, the reference values for the air-fuel ratio and the fuel injection rate may also be set to other values.

The air-fuel ratio feedback control is conducted on a per-bank basis (in other words, for each bank). For example, the detected values from the pre-catalyst sensor 20 and the post-catalyst sensor 21 on the left bank BL side are used only for the air-fuel ratio feedback control of the cylinders #1, #3, #5, and #7 belonging to the left bank BL, and are not used for the air-fuel ratio feedback control of the cylinders #2, #4, #6, and #8 belonging to the right bank BR. The reverse is similar. The air-fuel ratio feedback control is executed as though there were two independent, straight 4-cylinder engines. Also, in the air-fuel ratio feedback control, the same control amount is uniformly used for each cylinder belonging to the same bank.

In addition, to give an example of the ignition sequence for an engine 1 equipped with the above cylinder array and a right-hand 2-plane crankshaft, the cylinder sequence is #1, #8, #7, #3, #6, #5, #4, #2, and the ignition interval is an equal interval every 90 degrees CA when viewing the engine as a whole.

However, when the right bank BR and the left bank BL are each viewed individually, the ignition intervals are both unequal intervals, and the intervals respectively differ for the right bank BR and the left bank BL. Herein, provided that 0 degrees is the point at which the cylinder #1 on the left bank BL is ignited, subsequently the cylinder #8 on the right bank BR is ignited after 90 degrees CA, and next the cylinder #7 on the left bank BL is ignited after 90 degrees CA, and then the cylinder #3 on the same left bank BL is ignited after 90 degrees CA. In this way, although ignition of each cylinder occurs every 90 degrees CA, the interval is not equal in the right bank BR and the left bank BL internally.

Now assume that a failure of the injector 2 or the like occurs, for example in some of the cylinders (particularly in one cylinder), and an air-fuel ratio imbalance occurs among the cylinders. For example, on the left bank BL, in some cases insufficient valve closure of an injector 2 may cause the fuel injection rate of the cylinder #1 to increase past the fuel injection rate of the other cylinders #3, #5, and #7, causing the air-fuel ratio of the cylinder #1 to shift farther to the rich side than the air-fuel ratio of the other cylinders #3, #5, and #7.

Even at this point, if a comparatively large correction value is applied by the above air-fuel ratio feedback control, in some cases it is possible to keep the air-fuel ratio of the total gas (the converged exhaust gas) supplied to the pre-catalyst sensor 20 at the stoichiometric rate. However, when viewed per-cylinder, the cylinder #1 is much richer than stoichiometric, while the cylinders #3, #5, and #7 are leaner than stoichiometric. Only the overall balance is stoichiometric, which is clearly not preferable from an emissions standpoint. Consequently, in the present embodiment, an apparatus that detects such a cylinder air-fuel ratio imbalance abnormality is provided.

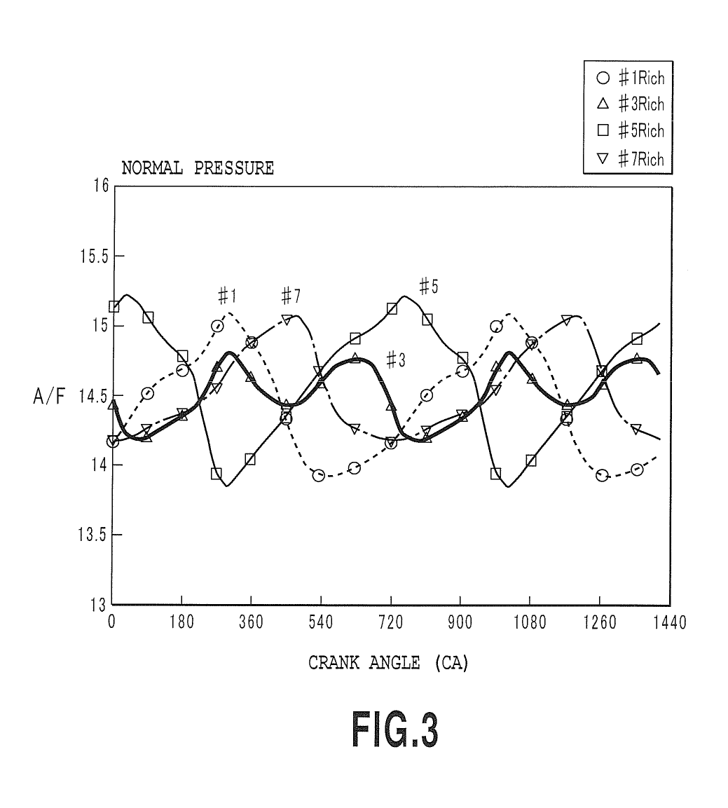

As illustrated in FIG. 3, the exhaust air-fuel ratio A/F detected by the pre-catalyst sensor 20 has a tendency to periodically fluctuate over a period of one engine cycle (720 degrees CA). In addition, if an air-fuel ratio imbalance among the cylinders occurs, the fluctuation within one engine cycle increases. FIG. 3 illustrates change in the air-fuel ratio detected by the pre-catalyst sensor 20 in the case where, under normal pressure, only cylinders #1, #3, #5, and #7 have an air-fuel ratio that is +50% rich, respectively, while the other three cylinders are stoichiometric. As illustrated, due to differences in the exhaust passage layout, the influences of air-fuel ratio imbalance differ for each cylinder.

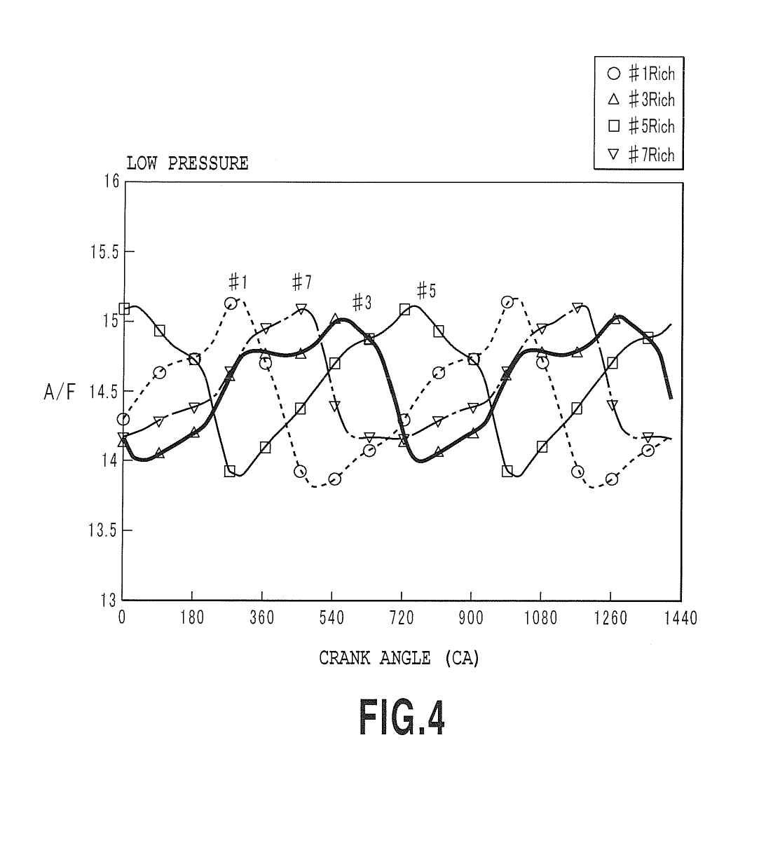

In addition, as illustrated for cylinder #3 only in FIG. 4, in a low-pressure environment (75 kPa, for example), the change in the air-fuel ratio in the case where the air-fuel ratio of only a specific cylinder is rich or lean exhibits a pattern that is different from the case of being under normal pressure (in this example in which only the cylinder #3 is +50% rich, the air-fuel ratio rises significantly near 300 to 700 degrees CA). This is thought to be caused because, in a low-pressure environment, blowdown gas (gas expelled from the time of opening the exhaust valve up until the piston reaches the bottom dead center) increases, and there is a lowered amount of squeezed gas when the piston rises up after having reached the bottom dead center. The influences of this air pressure also differ for each cylinder, and this is thought to be caused because the degree of interference of exhaust gas until reaching the pre-catalyst sensor 20 differs according to the geometrical shape of the exhaust passage and the ignition interval.

As the above description demonstrates, if an air-fuel ratio imbalance abnormality occurs, the fluctuation in the output of the pre-catalyst sensor 20 increases. Thus, by monitoring the degree of fluctuation, it is possible to detect an air-fuel ratio imbalance abnormality. In the present embodiment, there is calculated a fluctuation parameter, which is a parameter that is correlated with the degree of fluctuation in the output of the pre-catalyst sensor 20. This fluctuation parameter is compared against a predetermined abnormality determination value to detect an imbalance abnormality.

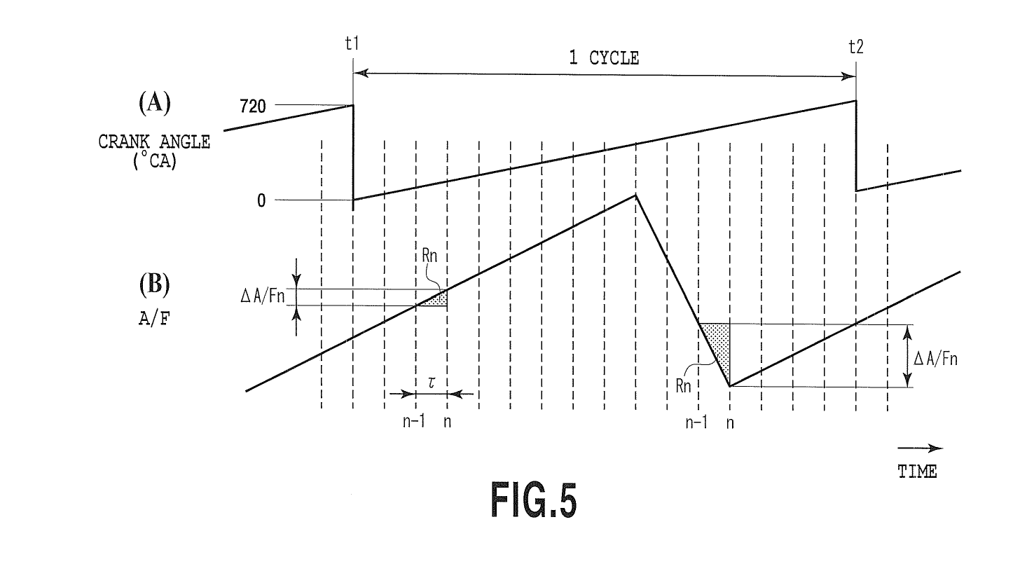

The method of calculating the fluctuation parameter will now be described. FIG. 5 is a timing chart that schematically illustrates a relationship between fluctuation in the output of the pre-catalyst sensor 20 within one engine cycle, and a fluctuation parameter. Herein, the value obtained by converting the output voltage Vf of the pre-catalyst sensor 20 into the air-fuel ratio A/F is used as the pre-catalyst sensor output. However, it is also possible to directly use the output voltage Vf of the pre-catalyst sensor 20.

As illustrated in FIG. 5(B), within one engine cycle, the ECU 100 acquires the value of the pre-catalyst sensor output A/F at a predetermined sample period τ (a unit of time, such as 4 ms, for example). The difference ΔA/Fₙ between the value A/Fₙ acquired at the current timing and the value A/Fₙ₋₁ acquired at the last timing is then calculated with the following Eq. 1. This difference ΔA/Fₙ may also be referred to as the derivative or the slope at the current timing.

ΔA/Fₙ=A/Fₙ−A/Fₙ₋₁ (1)

This difference ΔA/Fₙ expresses the fluctuation of the pre-catalyst sensor output in the simplest way. As the degree of fluctuation increases, the absolute value of the slope of the air-fuel ratio chart increases, because the absolute value of the difference ΔA/Fₙ increases. Accordingly, it is also possible to treat the value of the difference ΔA/Fₙ at one predetermined timing as the fluctuation parameter.

However, in order to increase precision in the present embodiment, the average value of multiple differences ΔA/Fₙ are treated as the fluctuation parameter. In the present embodiment, within one engine cycle, differences ΔA/Fₙ at respective timings are totaled, and the final total value is divided by the number of samples N to calculate the average value of the difference ΔA/Fₙ within one engine cycle. Furthermore, average values of the difference ΔA/Fₙ are totaled for M engine cycles (where M=100, for example), and the final total value is divided by the number of cycles M to calculate the average value ΔA/F_{AV} of the difference ΔA/Fₙ within M engine cycles, which is treated as the fluctuation parameter.

As the degree of fluctuation in the pre-catalyst sensor output increases, the absolute value of the average value of average values ΔA/F_{AV} within M engine cycles also increases. Thus, if the absolute value of that average value ΔA/F_{AV} is equal to or greater than a predetermined abnormality determination value, it is determined that there is an imbalance abnormality, where if that average value ΔA/F_{AV} is less than the abnormality determination value, it is determined that there is no imbalance abnormality, or in other words, that the system is normal.

Note that since the pre-catalyst sensor output A/F increases in some cases and decreases in some cases, the above difference ΔA/Fₙ or their average value ΔA/F_{AV} may be calculated for just one of these cases, and treated as the fluctuation parameter. Particularly in the case where only one cylinder is shifted to rich, the output when the pre-catalyst sensor receives exhaust gas corresponding to that one cylinder rapidly changes to the rich side (in other words, drops sharply), and thus it is possible to use decreasing values only for detecting rich shift (rich imbalance determination). In this case, only the region in the lower-right of the graph in FIG. 5(B) is used to detect rich shifts. Typically, since going from lean to rich is often sharper than going from rich to lean, with this method, precise detection of rich shifts may be anticipated. However, the configuration is not limited thereto, and it is also possible to use increasing values only, or alternatively, both decreasing and increasing values (by totaling absolute values of the difference ΔA/Fₙ and comparing this total value to a threshold value).

Also, any value that is correlated with the degree of fluctuation in the pre-catalyst sensor output may be treated as the fluctuation parameter. For example, it is also possible to calculate the fluctuation parameter on the basis of the difference between the maximum value and the minimum value (otherwise called the peak to peak) of the pre-catalyst sensor output within one engine cycle. This is because such a difference also increases as the degree of fluctuation in the pre-catalyst sensor output increases.

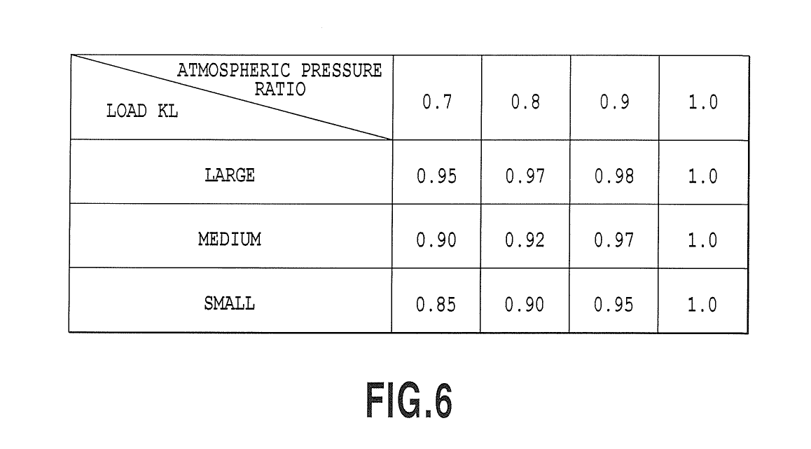

However, as discussed earlier, in a low-pressure environment, the change in the air-fuel ratio in the case in which the air-fuel ratio of only a specific cylinder is rich or lean (FIG. 4) exhibits a different pattern than the case of being under normal pressure (FIG. 3). Also, the degree of exhaust interference also changes depending on the load. For this reason, when detecting an air-fuel ratio imbalance abnormality, there is a risk that the detection precision may decrease due to the influences of atmospheric pressure and load, and misdetection may occur. Taking such phenomena into account, in the present embodiment, in order to correct the fluctuation parameter with consideration for the influences of atmospheric pressure and load, a correction coefficient map like that illustrated in FIG. 6 is created in advance and stored in the ROM of the ECU 100.

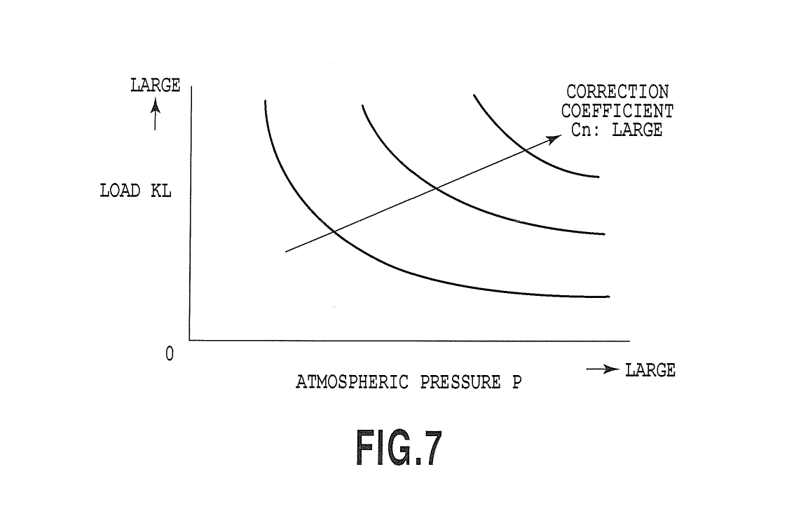

In the correction coefficient map in FIG. 6, a correction coefficient Cn to be multiplied by the fluctuation parameter (that is, the average value ΔA/F_{AV}) is determined as a function of the atmospheric pressure P and the load KL, such that the correction coefficient Cn increases as the atmospheric P increases, and as the load KL increases, to gradually approach 1. The atmospheric pressure P is expressed as a ratio (that is, an atmospheric pressure ratio) to normal pressure, such as 101.3 kPa, for example. This relationship may be generally illustrated as in the graph in FIG. 7. Note that if an abnormality threshold value α is to be corrected based on the atmospheric pressure P and the load KL, a correction coefficient to be multiplied by the abnormality threshold value α can be determined using a map represented by the same graph as FIG. 7, except that an indication “CORRECTION COEFFICIENT Cn: LARGE” in FIG. 7 is replaced by “CORRECTION COEFFICIENT Cn: SMALL”.

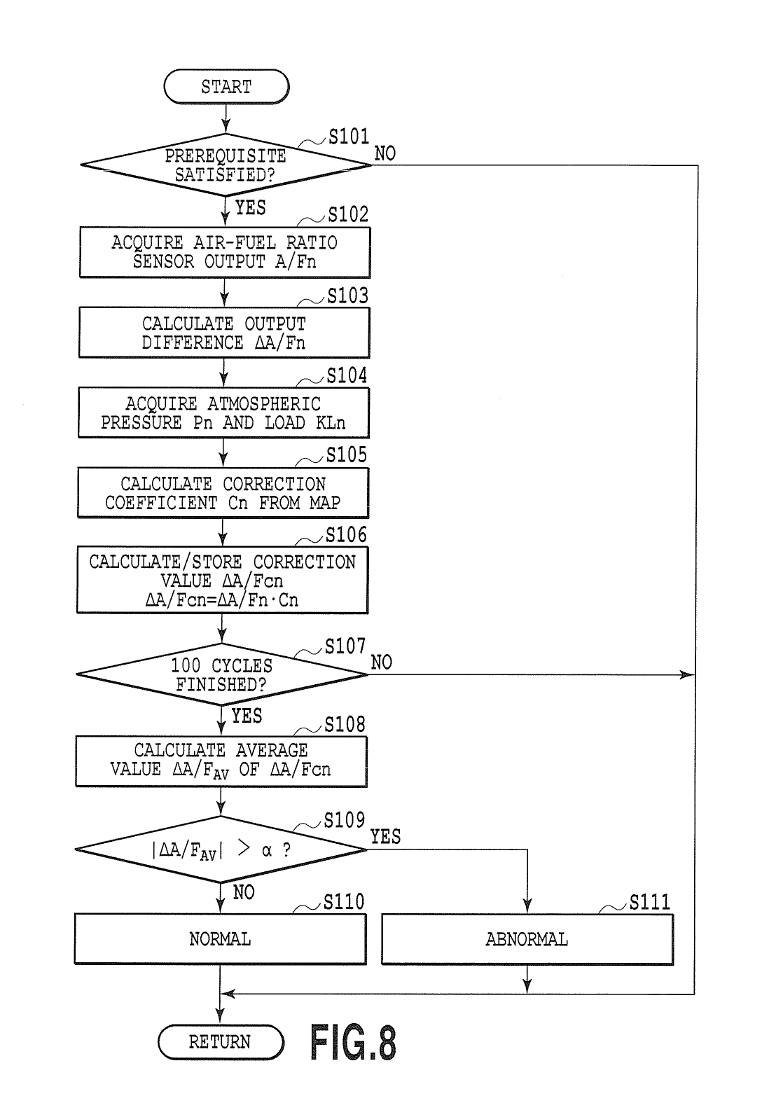

Next, FIG. 8 will be used to describe a cylinder air-fuel ratio imbalance abnormality detection routine. This routine is repeatedly executed by the ECU 100 at the above sample period τ, for example.

First, in step S101, it is determined whether or not a predetermined prerequisite suitable for conducting abnormality detection has been satisfied. This prerequisite is satisfied when all of the following conditions have been satisfied.
(1) Engine warm-up is finished. For example, warm-up is finished when the water temperature detected by the water temperature sensor 24 is equal to or greater than a predetermined value.
(2) At least the pre-catalyst sensor 20 is activated.
(3) The engine is in steady-state operation.
(4) Stoichiometric control is active.
(5) The engine is running within a detection region.
(6) The output A/F of the pre-catalyst sensor 20 is decreasing.

Of these, (6) indicates that the routine is based on the rich imbalance determination discussed earlier (the method that uses only decreasing values to detect rich shifts). The routine is ended in the case where the prerequisite is not satisfied. On the other hand, in the case where the prerequisite is satisfied, in step S102, the output A/Fₙ of the pre-catalyst sensor 20 (air-fuel ratio sensor) at the current timing is acquired, and in step S103, the output difference ΔA/Fₙ at the current timing is calculated with the earlier Eq. 1.

Next, in step S104, the atmospheric pressure Pn and the load KLn at the current timing are acquired. The atmospheric pressure Pn is acquired on the basis of a signal from the atmospheric pressure sensor 25. The atmospheric pressure Pn may also be acquired by an estimation calculation based on the throttle position and the airflow rate passing through the airflow meter. The load KLn is acquired on the basis of a signal from the accelerator position sensor 23, for example. The load KLn may also be acquired on the basis of another signal, such as a signal from the airflow meter 11, for example.

Next, in step S105, a correction coefficient Cn corresponding to the acquired atmospheric pressure Pn and the load KLn is calculated from the pre-created correction coefficient map (see FIGS. 6 and 7). As illustrated in FIGS. 6 and 7, the correction coefficient Cn is set to take a large value and approach 1 as the atmospheric pressure value Pn rises and the load KLn increases.

Next, in step S106, the output difference ΔA/Fₙ is corrected by multiplying the output difference ΔA/Fₙ at the current timing by the correction coefficient Cn at the current timing, and the corrected value ΔA/F_{cn} is calculated and stored in a predetermined storage area of the ECU 100.

Next, in step S107, it is determined whether the above process has finished 100 cycles. In the case of a negative determination, the above process is repeatedly executed until 100 cycles finish.

In the case in which 100 cycles have finished, in step S108, the average value ΔA/F_{AV} of the corrected values ΔA/F_{cn} calculated up to this point is calculated by dividing the total value of the corrected values ΔA/F_{cn} by the number of samples N and the number of engine cycles M, for example.

Then, in step S109, it is determined whether or not the absolute value of the average value ΔA/F_{AV} of the corrected values ΔA/F_{cn} is greater than a predetermined abnormality threshold value α. In the case where the absolute value ΔA/F_{AV} of the corrected values is less than the abnormality threshold value α, the routine proceeds to step S110, it is determined that there is no imbalance abnormality, or in other words that the system is normal, and the routine ends.

On the other hand, in the case where the absolute value ΔA/F_{AV} of the corrected values is equal to or greater than the abnormality threshold value α, the routine proceeds to step S111, it is determined that there is an imbalance abnormality, or in other words that there is a abnormality, and the routine ends. Note that, at the same time as determining abnormality, or in the case in which abnormality determination is successively returned for two trips (in other words, two consecutive trips, in which one trip lasts from engine start to stop), it is preferable to activate a warning device such as a check lamp to inform the user of the fact of the abnormality, and in addition, store the abnormality information in predetermined diagnosis memory in a form capable of being called by a maintenance worker.

As thus explained, in the present embodiment, the value of an average value ΔA/F_{AV}, which is used as a fluctuation parameter correlated with the degree of fluctuation in the output of an air-fuel ratio sensor 20, is compared to an abnormality threshold value α, and in order to detect a cylinder air-fuel ratio imbalance abnormality, the value of the output difference ΔA/Fₙ used as the fluctuation parameter is corrected on the basis of the atmospheric pressure Pn, while an amount of correction (a correction coefficient Cn) is modified according to the load KLn. In this way, since the amount of correction is modified according to the load, the detection precision is improved while taking into account the influences of the load, and misdetections may be suppressed.

Next, a second exemplary embodiment of the present invention will be described. In the above first embodiment, a shared correction coefficient map is used for all cylinders, but instead of such a configuration, in the second embodiment, multiple types of mutually differing correction coefficient maps are used, and mutually differing types of correction coefficient maps are used for one part and another part of the multiple cylinders.

Particularly, in the second embodiment, individual correction coefficient maps are used for the multiple cylinders. In other words, a correction coefficient map as illustrated by example in FIGS. 6 and 7 is created in advance for each of the cylinders #1 to #8 and stored in the ROM of the ECU 100, with the value of the correction coefficient Cn being made to mutually differ in these respective maps. The remaining mechanical configuration in the second embodiment is similar to the above first embodiment, and thus detailed description thereof will be reduced or omitted.

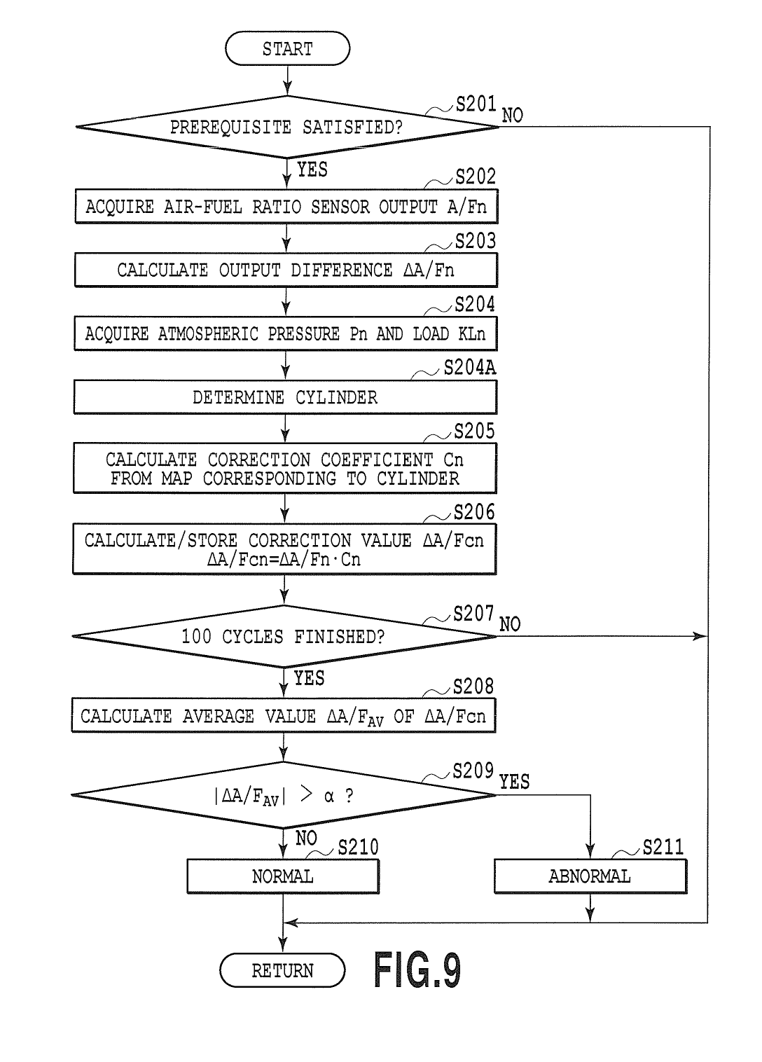

A cylinder air-fuel ratio imbalance abnormality detection routine according to the second embodiment will now be described using FIG. 9.

The determination of whether or not a predetermined prerequisite is satisfied in step S201, the acquisition of the output A/Fₙ of the pre-catalyst sensor 20 (air-fuel ratio sensor) in step S202, the calculation of the output difference ΔA/Fₙ in step S203, and the acquisition of the atmospheric pressure Pn and the load KLn in step S204 are respectively similar to steps S101 to S104 in the foregoing first embodiment.

In step S204A, the cylinder whose exhaust gas corresponds to the currently detected air-fuel ratio is determined. This determination is conducted on the basis of a signal from the crank position sensor 22, while taking into account a predetermined delay time (for example, by adding a correction according to a signal from the airflow meter 11).

Next, in step S205, a correction coefficient Cn corresponding to the acquired atmospheric pressure Pn and the load KLn is calculated from a correction coefficient map corresponding to that cylinder. In other words, from among the multiple correction coefficient maps created in advance for each of the cylinders #1 to #8 and stored in the ROM of the ECU 100, the one corresponding to the cylinder determined in step S204A is selected and used, and a correction coefficient Cn is calculated thereby.

The processing in steps S206 to S211 is similar to the processing in steps S106 to S111 in the foregoing first embodiment.

As a result of the above process, in the second embodiment, the output difference ΔA/Fₙ used as a fluctuation parameter is corrected on the basis of the atmospheric pressure Pn, while an amount of correction (a correction coefficient Cn) is modified according to the load KLn. In this way, since the amount of correction is modified according to the load, the detection precision is improved while taking into account the influences of the load, and misdetections may be suppressed.

Also, given that the influences on the degree of exhaust interference due to changes in the load differs for each cylinder, in the second embodiment, multiple, mutually differing correction coefficient maps are provided, and mutually differing correction coefficient maps are applied to one part and another part of the multiple cylinders in order to determine an amount of correction. Consequently, since the amount of correction is determined by a different correction coefficient for each cylinder, the detection precision is improved, and misdetections may be suppressed.

Note that although a different correction coefficient map is used for each of the cylinders #1 to #8 in the second embodiment, the number of types of correction coefficient maps is not required to be the same number as the number of cylinders, as long as there are a plurality of types. For example, it is possible to use a shared correction coefficient map for a plurality of cylinders having a mutually similar geometrical shape or layout of the exhaust passage up to the air-fuel ratio sensor. Also, in the case in which the geometrical shape or layout of the exhaust passage is symmetric or approximately symmetric between multiple banks or cylinder groups, for the multiple cylinders in a symmetric relationship (for example, cylinders #1 and #2, #3 and #4, #5 and #6, and #7 and #8 in a V8 engine like in the second embodiment), respectively shared (that is, a total of four types of) correction coefficient maps may be used.

The above thus describes preferred embodiments of the present invention in detail, but various other embodiments of the present invention are also conceivable. For example, in the foregoing embodiments, a rich shift abnormality is detected by using only the decreasing (changing to the rich side) air-fuel ratio sensor output. However, a configuration that uses only the increasing (changing to the lean side) air-fuel ratio sensor output, or a configuration that uses both the decreasing and the increasing air-fuel ratio sensor output, is also possible. Also, it is possible to detect not only rich shift abnormality but also lean shift abnormality, and air-fuel ratio imbalance abnormality may also be broadly detected without distinguishing between rich shifts and lean shifts.

Also, although the foregoing embodiments correct the value of the fluctuation parameter, the abnormality threshold value α may also be corrected according to the atmospheric pressure P and the load KL. In the case of correcting the abnormality threshold value α, it is suitable to modify a correction coefficient by which to multiply the abnormality threshold value α in a direction such that the absolute value of the degree of fluctuation decreases as the atmospheric pressure P increases, and as the load KL increases. Furthermore, both the fluctuation parameter and the abnormality threshold value may also be corrected on the basis of the atmospheric pressure P and the load KL.

Also, in the foregoing embodiments, the example of detecting a rich shift abnormality was primarily described in order to ease understanding. However, the present invention is also applicable to the case of detecting a lean shift abnormality. The present invention is not limited to a V8 engine, and is also applicable to a V-type engine with a different cylinder count (such as 6-cylinder, 10-cylinder, or 12-cylinder, for example), an internal combustion engine having a plurality of cylinder groups such as a horizontally opposed engine, or an inline engine.

An embodiment of the present invention is not limited to the foregoing embodiments and their modifications, and all such modifications and applications or their equivalents that are encompassed by the ideas of the present invention as stipulated by the claims are to be included in the present invention. Consequently, the present invention is not to be interpreted in a limited manner, and is also applicable to other arbitrary technologies belonging within the scope of the ideas of the present invention.

What is claimed is:

1. An apparatus for detecting cylinder air-fuel ratio imbalance abnormality of a multi-cylinder internal combustion engine, comprising:
one or a plurality of air-fuel ratio sensors installed in an exhaust passage of the multi-cylinder internal combustion engine;
an abnormality detecting unit that detects a cylinder air-fuel ratio imbalance abnormality by comparing a value of a parameter correlated with a degree of fluctuation in the air-fuel ratio sensor output to an abnormality threshold value; and
a correcting unit that corrects at least one of the value of the parameter or the abnormality threshold value on the basis of atmospheric pressure;
wherein an amount of correction performed by the correcting unit is modified according to a load of the multi-cylinder internal combustion engine.

2. The apparatus according to claim 1, wherein the correcting unit corrects the value of the parameter, while the amount of correction is modified in a direction such that an absolute value of the degree of fluctuation increases as the load increases.

3. The apparatus according to claim 1, wherein the correcting unit corrects the value of the abnormality threshold value, while the amount of correction is modified in a direction such that an absolute value of the degree of fluctuation decreases as the load increases.

4. The apparatus according to claim 1, wherein a plurality of mutually differing correcting units are provided, and the mutually differing correcting units are applied to one part and another part of a plurality of cylinders.
